(19) United States
(12) Patent Application Publication
(10) Pub. No.: US 2023/0172769 A1
(43) Pub. Date: Jun. 8, 2023

(54) Absorbent Garment with Softened Waistband

(71) Applicant: Kimberly-Clark Worldwide, Inc., Neenah, WI (US)

(72) Inventors: Kai Cui, Shanghai (CN); Tongtong Zhang, Beijing (CN); JungSun Lim, Reigate (GB); Vadim Biryukov, Stupino (RU); Lixiang Ji, Nanjing (CN); Jiajun Yang, Shanghai (CN); Daniel Cote, Neenah, WI (US)

(21) Appl. No.: 17/923,933

(22) PCT Filed: Jun. 28, 2020
(86) PCT No.: PCT/CN2020/098448

§ 371 Date: Nov. 8, 2022

(51) Int. Cl. A61F 13/513; A61F 13/496

(52) U.S. Cl. CPC A61F 13/513; A61F 13/496

(57) ABSTRACT

An absorbent article (10) includes a longitudinal axis (30) and a lateral axis (32). The absorbent article (10) is a pant that includes a front region (12), a back region (14) and a crotch region (16) disposed between the front region (12) and the back region (14). The crotch region (16) is formed by an absorbent assembly (34). In addition to the absorbent assembly (34), the absorbent article (10) includes a front panel (57) and a back panel (61); the front panel (57) is attached to a front region of the absorbent assembly (34) and the back panel (61) is attached to a back region of the absorbent assembly (34). The front panel (57) and the back panel (61) include a softened material layer (205). The absorbent article (10) has a waistband that provides a custom fit and is soft and comfortable during wear, such that irritation to the skin is reduced or eliminated.

### BACKGROUND

Absorbent articles, such as diapers, incontinence garments, training pants, sanitary napkins, panty liners, and the like are well known in the art. These articles, which are often disposable, are capable of absorbing and retaining fluids and other bodily discharges. Some absorbent articles, such as pull-on type absorbent articles, include a central absorbent member and side panels extending from and interconnecting respective front and back regions of the absorbent member forming a waist assembly.

An example of a pull-on absorbent article is a diaper pant; diaper pants are typically worn by babies who are able to stand up, but who are not yet old enough to be toilet trained. Absorbent articles in the diaper pants format typically include a waist assembly (that includes side panels) formed of elastic strands or other elastic materials placed between inner and outer layers of a nonwoven material. The elastic strands are typically kept in place by adhesive. These types of waist assemblies may include 20 to 40 high-tension elastic strands that embrace the waist area. The use of the elastic strands in the side panels of the diaper pants allows the side panels to be highly elastic and allows for a wide range of body shapes over which the diaper pant may be donned. The elastic side panels also provide a conforming fit for the wearer.

Such elastic side panels in traditional diaper pants can cause irritation to a baby's skin during use. A desirable feature of a diaper pant is to have a soft and comfortable waistband or waist area—one that does not put undue pressure on the skin, which could lead to red-marking of the skin.

Accordingly, there remains a need for a diaper pant having a waistband that provides a “custom” fit and is soft and comfortable during wear, such that irritation to the wearer's skin is reduced or eliminated.

### SUMMARY

The present invention is directed to an absorbent garment that provides a solution for a more comfortable waistband that can reduce irritation for the wearer. The absorbent garments of the invention have a longitudinal direction and a lateral direction. The lateral direction is generally perpendicular to the longitudinal direction and describing the absorbent garments with these orientations improves the understanding of the components of the garment in relation to one another. The absorbent garments also have a central lateral axis extending in the lateral direction about the center of the garment 10. The absorbent garments include a front region, a back region and a crotch region—where the crotch region is disposed between the front region and the back region. The crotch region includes an absorbent assembly. The absorbent assembly has a front edge, a back edge and a pair of laterally-opposed side edges (that is, side edges that are opposite each other in the lateral direction). The absorbent assembly includes a skin-facing liner and an absorbent composite. The absorbent garments also include a front panel in the front region and a back panel in the back region. Both the front panel and the back panel include an inner layer, an outer layer, and a softened material layer. The front panel and back panel also include an elastic area that is disposed between the inner layer and the outer layer. The front panel and the back panel are bonded or otherwise joined together to form side seams of the absorbent garment. A portion of the front panel and the back panel are folded over forming a front waist edge and a back waist edge of the garment. The folded over portion of the front panel includes a front fold line having a portion of the softened material layer folded over the front fold line. The folded over portion of the front panel includes a first attachment region bonding the outer layer to the skin-facing liner and a second attachment region bonding the softened material layer to the outer layer. The folded over portion of the back panel includes a back fold line having a portion of the softened material layer folded over the back fold line. The folded over portion of the back panel includes a third attachment region bonding the outer layer to the skin-facing liner and a fourth attachment region bonding the softened material layer to the outer layer.

In another aspect, provided is a garment having a front panel in the front region and a back panel in the back region. The front panel includes an inner layer, an outer layer, a softened material layer, and an elastic area disposed between the inner and outer layer. The softened material layer is disposed between the elastic area and the outer layer. A portion of the front panel is folded over forming the front waist edge of the garment. The front panel includes a first attachment region bonding the outer layer to the skin-facing liner and a second attachment region bonding the softened material layer to the outer layer. The back panel includes an inner layer, an outer layer, a softened material layer, and an elastic area disposed between the inner and outer layer. The softened material layer is disposed between the elastic area and the outer layer. A portion of the back panel is folded over forming the back waist edge of the garment. The back panel includes a third attachment region bonding the outer layer to the skin-facing liner and a fourth attachment region bonding the softened material layer to the outer layer.

In another aspect, provided is a garment having a front panel in the front region and a back panel in the back region. The front panel includes an inner layer, an outer layer, a softened material layer, and an elastic area disposed between the inner and outer layer. The inner layer includes a first end comprising a softened material layer. The first end including the softened material layer is folded over the elastic area such that the softened material layer forms a folded over portion disposed between the elastic area and the outer layer. A portion of the front panel is folded over forming the front waist edge of the garment. The outer layer can be attached to the skin-facing liner at a first attachment region. The back panel includes an inner layer, an outer layer, a softened material layer, and an elastic area disposed between the inner and outer layer. The inner layer includes a second end comprising a softened material layer. The second end including the softened material layer is folded over the elastic area such that the softened material layer forms a folded over portion disposed between the elastic area and the outer layer. A portion of the back panel is folded over forming a back waist edge of the garment. The outer layer can be attached to the skin-facing liner at a second attachment region.

The present disclosure will be more fully understood, and further features will become apparent, when reference is made to the following detailed description and the accompanying drawings.

### BRIEF DESCRIPTION OF THE DRAWINGS

A full and enabling disclosure of the present disclosure is set forth more particularly in the remainder of the specification, including reference to the accompanying figures, in which:

FIG. 1 is a top down view of an absorbent garment of the present disclosure, in an unassembled condition;

FIG. 2 is a front view of the absorbent garment of FIG. 1, in an assembled condition;

FIG. 3 is a top down view of the back region of an absorbent garment of the present disclosure;

FIG. 4 is a top down view of the front region of an absorbent garment of the present disclosure;

FIG. 5 is a lengthwise cross-sectional view of an absorbent garment of the present disclosure;

FIG. 6 is a lengthwise cross-sectional view of an absorbent garment of the present disclosure;

FIG. 7 is a lengthwise cross-sectional view of an absorbent garment of the present disclosure;

FIG. 8 is a lengthwise cross-sectional view of an absorbent garment of the present disclosure;

FIG. 9 is a lengthwise cross-sectional view of an absorbent garment of the present disclosure; and

FIG. 10 is a lengthwise cross-sectional view of an absorbent garment of the present disclosure.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

The term “absorbent garment” or “absorbent article” refers herein to a garment that can be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent garments, including, but not limited to, infant diaper pants, toilet-training pants, youth pants, swim pants, feminine hygiene garments and adult incontinence garments. The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements.

The term “carded web” refers herein to a web containing natural or synthetic staple fibers typically having fiber lengths less than about 100 mm. Bales of staple fibers can undergo an opening process to separate the fibers that are then sent to a carding process that separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers can be subject to adhesive processes to bind the fibers together such as by the use of powder adhesives. The carded web can be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films that constitute liquid transfer films, as well as films that do not transfer fluids, such as, but not limited to, barrier films, filled films, breathable films, and oriented films.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable. The term “meltblown” refers herein to fibers formed by extruding a molten thermoplastic material through a plurality of fine, usually circular, die capillaries as molten threads or filaments into converging high velocity heated gas (e.g., air) streams that attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers that can be continuous or discontinuous, are generally smaller than about 0.6 denier, and can be tacky and self-bonding when deposited onto a collecting surface. The term “nonwoven” refers herein to materials and webs of material that are formed without the aid of a textile weaving or knitting process. The materials and webs of materials can have a structure of individual fibers, filaments, or threads (collectively referred to as “fibers”) that can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials that are compliant and that will readily conform to the general shape and contours of the wearer's body.

The term “spunbond” refers herein to small diameter fibers that are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an aspect, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an aspect, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a polymeric material that becomes pliable or moldable above a specific temperature and returns to a solid state upon cooling.

The term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configuration of the material. These configurations include, but are not limited to isotactic, syndiotactic and random symmetries.

The term “softened material layer” refers herein to a polymeric material that has been treated to increase the softness of the material. The treatment can include any mechanical or chemical process known to increase the softness of a material. Such a softened material layer may be generally formed from any suitable nonwoven material. In at least some embodiments, the softened material layer may comprise a spunmelt nonwoven material comprising randomly laid continuous filaments. The softened material layer may also be a spunbond nonwoven material or a through air bonded, carded web (TABCW) in other embodiments. Further aspects of the softened material layers of the present disclosure are described in more detail below.

Generally, an absorbent garment having a softened waistband area is disclosed herein. The primary purpose of the absorbent garment is to provide additional comfort when worn while also ensuring a custom fit around the waist of the wearer. Pant format absorbent garments, such as those of the present disclosure, are typically designed and constructed to fit a wide range of body shapes and to be comfortable for the user to wear. A common pant construction includes a generally rectangular absorbent assembly integrated with a front waist panel and a back waist panel. The waist panels typically include elastic material, such as stranded elastics, and it is the working combination of the front panel and the back panel that provides the fit range of the pant. With the absorbent pant garments of the present disclosure, the lateral direction edges of the front panel and the back panel are bonded together to form the side seams of the garment. Advantageously, the pant garments of the present disclosure include a softened material layer disposed on the front waist panel and the back waist panel such that garment provides an extra-soft feeling around the waist opening of the garment. Furthermore, the softened material is bonded to the front waist panel and back waist panel in such a manner as to not diminish the softness of the softened material layer.

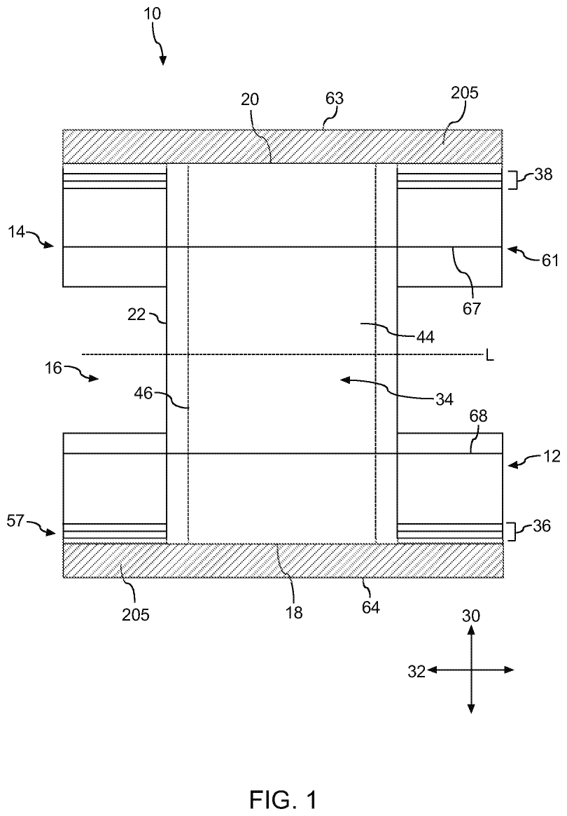

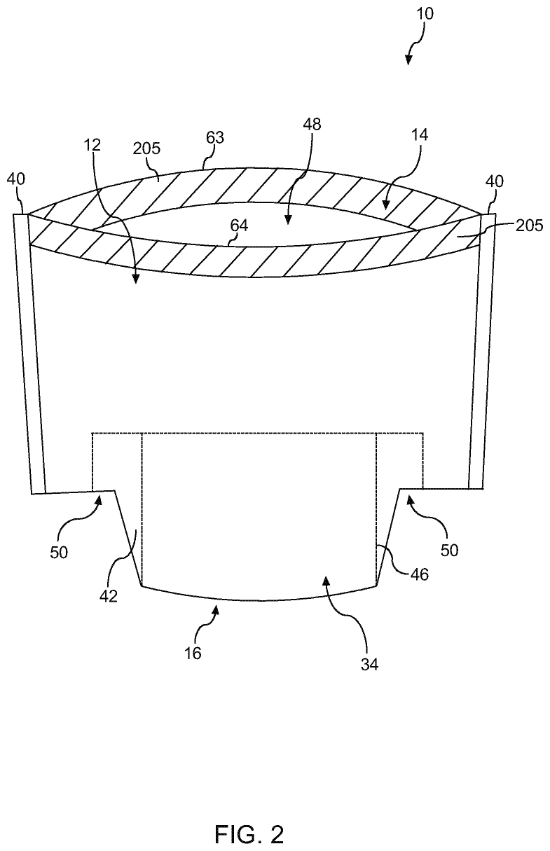

With reference to the drawings, FIGS. 1 and 2 illustrate one suitable aspect of an absorbent garment of the present disclosure in the form of a diaper pant, indicated generally at 10. While the present disclosure will be made in the context of the diaper pant 10, it should be understood that aspects of the present disclosure are applicable to other absorbent garments, such as, for example open diaper articles, toilet-training pants, youth pants, swim pants, feminine hygiene garments and adult incontinence garments. As used herein, the term “absorbent garment” refers to garments that absorb and contain body exudates and that are intended to be discarded after a limited period of use. The garments are not intended to be laundered or otherwise restored for reuse. Put differently, the garments are disposable. The garments can be placed against or in proximity to the body of a wearer to absorb and contain various exudates discharged from the body.

FIG. 1 illustrates the absorbent garment, such as a diaper pant, 10 in an unfolded and laid flat condition to show an inner surface of the absorbent garment that faces the wearer when the absorbent garment 10 is worn. The absorbent garment 10 has a longitudinal axis (direction) 30 and a lateral axis (direction) 32. Along the longitudinal axis 30, the absorbent garment 10 defines a front region 12, a back region 14, and a crotch region 16 extending between and connecting the front region 12 and the back region 14. The absorbent garment 10 also has a central lateral axis “L” extending in the lateral axis direction 32 about the center of the garment 10.

The absorbent garment 10 includes a central absorbent assembly, indicated generally at 34, that extends longitudinally from the front region 12 through the crotch region 16 to the back region 14. The central absorbent assembly 34 of the illustrated aspect includes an outer cover 42 (shown in FIG. 2) forming an outward facing surface of the garment 10 and a skin-facing (or “bodyside”) liner 44 connected to the outer cover 42 in a superposed relation by suitable means such as adhesives, ultrasonic bonds, thermal bonds, pressure bonds, or combinations thereof. The outer cover 42 can be a multilayer material. An absorbent composite 46 is disposed between the outer cover 42 and the bodyside liner 44. The absorbent assembly 34 also includes a front edge 18, a back edge 20 and a pair of laterally-opposed side edges 22, 24. The front and back regions 12, 14 of the absorbent garment 10 include a front panel 57 and a back panel 61, respectively. The front panel 57 and the back panel 61 are interconnected via at least the absorbent assembly 34. The front panel 57 is a laminate or composite of several materials including an inner layer, and outer layer, and a softened material layer 205. Similarly, the back panel 61 is also a laminate or composite of several materials including an inner layer, outer layer, and softened material layer. Between the inner layer and outer layer of the front panel 57, there is an elastic area 36. Between the inner layer and the outer layer of the back panel 61 there is an elastic area 38.

With respect to the front region 12 of the absorbent garment 10, a portion of the front panel 57 is folded over defining the front fold line 64 at the front waist edge of the garment 10. The folded over portion of the front panel 57 can be secured to the body facing side of the garment 10. For example, the folded over portion of the front panel 57 can be secured to the inner layer of the front panel 57. In certain embodiments, the folded over portion of the front panel 57 can be secured to the absorbent assembly. The folded over portion of the front panel 57 includes at least a portion of the outer layer 202. In at least some embodiments, the folded over portion of the front panel 57 may also include a portion of the inner layer 200 and/or the softened material layer 205. Although in such embodiments, the outer layer 202 may fully cover the inner layer when viewing the body facing side of the garment 10.

With respect to the back region 14 of the absorbent garment 10, a portion of the back panel 61 is folded over defining the back fold line 63 at the back waist edge of the garment 10. The folded over portion of the back panel 61 can be secured to the body facing side of the garment 10. For example, the folded over portion of the back panel 61 can be secured to the inner layer of the back panel 61. In certain embodiments, the folded over portion of the back panel 61 can be secured to the absorbent assembly 34. Similar to the front panel 57, the folded over portion of the back panel 61 includes at least a portion of the outer layer 202. In at least some embodiments, the folded over portion of the back panel 61 may also include a portion of the inner layer 200 and/or the softened material layer 205. Although in such embodiments, the outer layer 202 may fully cover the inner layer when viewing the body facing side of the garment 10.

As shown in FIG. 1, the front panel 57 includes a pair of laterally-opposed, front side portions extending outward from the lateral center of the absorbent garment 10. Likewise, the back panel 61 incudes a pair of laterally-opposed, back side portions extending outward from the lateral center of the absorbent garment 10. As shown in FIG. 2, the front panel 57 is joined to the back panel 61 by a pair of butt (or fin) side seams 40 to define a pull-on, pant-like configuration of the absorbent garment 10 having a waist opening, indicated at 48, and two leg openings, indicated at 50. More specifically, each front side portion is joined to a respective back side portion via one of the butt side seams 40.

With the absorbent garment 10 in the pull-on, pant-like configuration, illustrated in FIG. 2, the front region 12 includes the portion of the absorbent garment 10 that, when worn, is positioned at least in part on the front of the wearer while the back region 14 includes the portion of the absorbent garment 10 that is positioned at least in part on the back of the wearer. The crotch region 16 of the absorbent garment 10 includes the portion of the absorbent garment 10 that is positioned between the legs of the wearer and covers the lower torso of the wearer. The front and back side portions include the portions of the absorbent garment 10 (and more particularly of the front and back regions 12, 14) that, when worn, are positioned on the hips of the wearer. As shown in FIG. 2, the front panel 57 and the back panel 61 cooperatively define the waist opening 48 of the absorbent garment 10 that is configured to fully encircle the waist of the wearer. Further, as shown in FIG. 2 each of the front panel 57 and the back panel 61 incorporate a softened material layer 205.

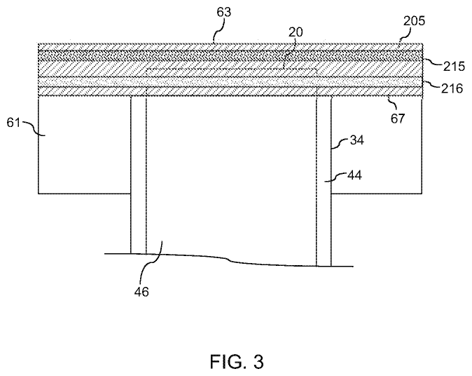

In one aspect of the present invention, and as illustrated in FIG. 3, the absorbent garment 10 includes a folded over portion of the back panel 61 that is folded over to form a back fold line 63 defining the back waist edge of the garment 10. In certain embodiments, the folded over portion of the back panel 61 can be secured to the body side of the garment 10. In certain embodiments, the folded over portion of the back panel 61 can be secured to the inner layer of the back panel 61. In other embodiments, the folded over portion of the back panel 61 can be secured to the absorbent assembly 34. In such an embodiment, the folded over portion of the back panel 61 can be bonded to the skin-facing liner 44 of the absorbent assembly 34. Between the back fold line 63 and the edge of the folded over portion 67, a softened material layer 205 faces the body side of the absorbent garment. The purpose of the softened material layer 205 is to provide enhanced comfort to the wearer, such that skin irritation can be reduced. In order to perform this function, in one embodiment the softened material layer 205 can be adhered to a portion of the outer layer 202 of the back panel 61 on the outward facing surface, such that when the back panel 61 is folded over to form the back waist edge along the back fold line 63, the softened material layer 205 includes a portion that faces the bodyside of the garment 10 and also extends over the fold back fold line 63. In such an embodiment, the folded over portion of the back panel 61 includes an attachment region 215 located on the body facing side of the garment 10 for bonding the softened material layer 205 to the outer layer of the folded over portion of the back panel 61. The back panel 61 can also include an attachment region 216 for bonding the outer layer 202 to the body facing side of the garment 10. In some embodiments, the attachment region 216 can bond the outer layer 202 to the skin-facing liner 44 of the absorbent assembly 34. In still other embodiments, the attachment region 216 can bond the outer layer 202 to the inner layer 200 of the back panel 61.

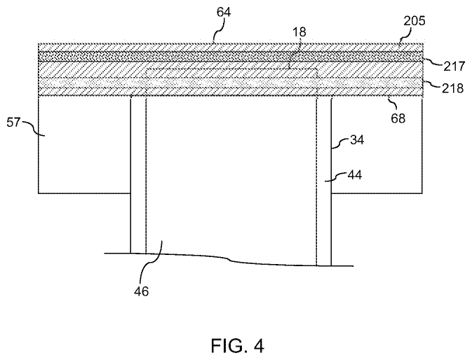

As illustrated in FIG. 4, a portion of the front panel 57 is folded over to form a front fold line 64 defining the front waist edge of the garment 10. In certain embodiments, the folded over portion of the front panel 57 can be secured to the body side of the garment 10. In certain embodiments, the folded over portion of the front panel 57 can be secured to the inner layer of the front panel 57. In other embodiments, the folded over portion of the front panel 57 can be secured to the absorbent assembly 34. In such an embodiment, the folded over portion of the front panel 57 can be bonded to the skin-facing liner 44 of the absorbent assembly 34. Between the front fold line 64 and the edge of the folded over portion 68, a softened material layer 205 is disposed on the body facing side of the absorbent garment 10. The purpose of the softened material layer 205 is to provide enhanced comfort to the wearer, such that skin irritation can be reduced. In order to perform this function, in one embodiment the softened material layer 205 can be adhered to a portion of the outer layer 202 of the front panel 57 on the outward facing surface, such that when the front panel 57 is folded over the absorbent assembly 34, the softened material layer 205 includes a portion that faces the bodyside of the garment 10 and also extends over the front fold line 64. In such an embodiment, the folded over portion of the front panel 57 includes an attachment region 217 located on the body facing side for bonding the softened material layer 205 to the outer layer 202 of the front panel 57. The front panel 57 can also include an attachment region 218 for bonding the outer layer 202 of the folded over portion of the front panel 57 to the body facing side of the garment 10. In some embodiments, the attachment region 218 can bond the outer layer 202 to the skin-facing liner 44 of the absorbent assembly 34. In still other embodiments, the attachment region 218 can bond the outer layer 202 to the inner layer 200 of the back panel 61.

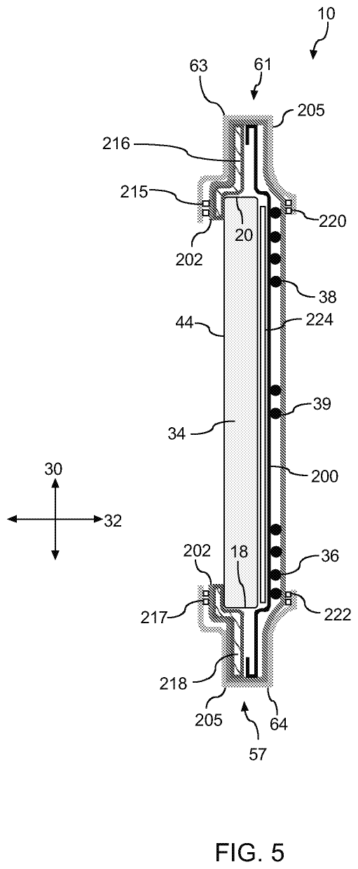

FIG. 5 depicts a longitudinal cross section of an example absorbent garment 10 according to various embodiments of the present disclosure. As shown, the garment 10 includes an absorbent assembly 34 having a skin-facing liner 44 thereon. For example, absorbent assembly 34 can include a skin-facing liner 44 on one side of the absorbent assembly 34 and be bonded to an inner layer 200 on the opposite side of the absorbent assembly 34. While one side of the inner layer 200 is bonded to the absorbent assembly 34, the other and opposite side of the inner layer 200 is coupled to an elastic area 36 and 38 disposed between the inner layer 200 and the outer layer 202. To form the back panel 61, a portion of the outer layer 202 is folded over forming a back fold line 63. The folded over portion of the back panel 61 forms the back waist edge of the garment 10 generally along the back fold line 63. The folded over portion of the back panel 61 is bonded to the skin-facing liner 44 at an attachment region 216. As shown, the attachment region 216 can be configured such that only the outer layer 202 is bonded to the skin-facing liner 44 at attachment region 216. In other embodiments, however, the attachment region 216 may bond a portion of both the outer layer 202 and the inner layer 200 to the skin-facing liner 44. To form the front panel 57, a portion of the outer layer 202 is folded over forming a front fold line 64. The folded over portion of the front panel 57 forms the front waist edge of the garment 10 generally along the front fold line 64. The folded over portion of the front panel 57 is bonded to the skin-facing liner 44 at an attachment region 218. As shown, the attachment region 218 can be configured such that only the outer layer 202 is bonded to the skin-facing liner 44 at attachment region 218. In other embodiments, however, the attachment region 218 may bond a portion of both the outer layer 202 and the inner layer 200 to the skin-facing liner 44.

As shown herein with respect to FIG. 5 and the other Figures, both the inner layer 200 and the outer layer may span between the front and back regions 12, 14 and through the crotch region 16. In other contemplated embodiments, however, the inner layer 200 in each of the front and back regions 12, 14 may be formed of separate, independent materials such that the inner layers 200 of the front and back regions are unconnected. Any of these embodiments where at least one layer spans between the front and back regions 12, 14 may generally be termed a “one-piece” garment or as having a “one-piece” construction in the art. In further embodiments contemplated by the present disclosure, each of the front panel 57 and the back panel 61 can comprise separate materials forming the outer layer of each panel 57, 61. For example, both of the inner layer 200 and the outer layer 202 of the front region 12 may be fully unconnected from the respective inner layer 200 and the outer layer 202 of the back region 14. In such embodiments, the assembly 34 may be the only portion of the garment connecting the front and back regions 12, 14. Any of these such embodiments may be termed “three-piece” garments or having a “three-piece” construction in the art.

A softened material layer 205 is disposed on the outer layer 202 such that a portion of the softened material layer 205 is folded over the back fold line 63 and the front fold line 64. Generally, the softened material layer 205 of each of the front and back regions 12, 14 may be separate materials coupled to the outer layer 202 in each of the regions 12, 14. Although, it is possible in other embodiments for the softened material layer 205 to comprise a single, unitary piece of material.

For the back panel 61, a portion of the softened material layer 205 can be bonded to the outer layer 202 at an attachment region 220 disposed on the outward facing surface, folded over the back fold line 63, and bonded to the outer layer 202 an attachment region 215 that is on the bodyside of the garment 10. Similarly, for the front panel 57, a portion of the softened material layer 205 can be bonded to the outer layer 202 at an attachment region 222 disposed on the outward facing surface, folded over the front fold line 64, and bonded to the outer layer 202 at an attachment region 217 that is on the bodyside of the garment 10. In certain embodiments, the softened material layer 205 remains unbonded from the outer layer 202 except with respect to attachment region 222 and 217. As shown, attachment regions 215 and 217 bond only a portion of the softened material layer 205 to the outer layer 202. In at least some of the embodiments, the softened material layer 205 is un-attached to the attachment regions 216 and/or 218. For example, no portion of the attachment regions 216 and/or 218 may be uncovered by the outer layer 202 such that the softened material layer 202 is coupled directly to the attachment regions 216 and/or 218.

The folded over portion of the back panel 61 can include a softened material layer 205 having an end along the bodyside of the garment that is disposed approximately an equal distance to the central lateral axis (L) as compared to the end of the outer material layer 202 along the bodyside of the garment. In other embodiments, the folded over portion of the back panel 61 can include a softened material layer 205 having an end along the bodyside of the garment that is disposed closer to the central lateral axis (L) as compared to the end of the outer material layer 202 along the bodyside of the garment. In still further embodiments, the folded over portion of the front panel 61 can include a softened material layer 205 having an end along the body side of the garment that is disposed further away from the central lateral axis (L) as compared to the end of the outer material layer 202 along the body side of the garment.

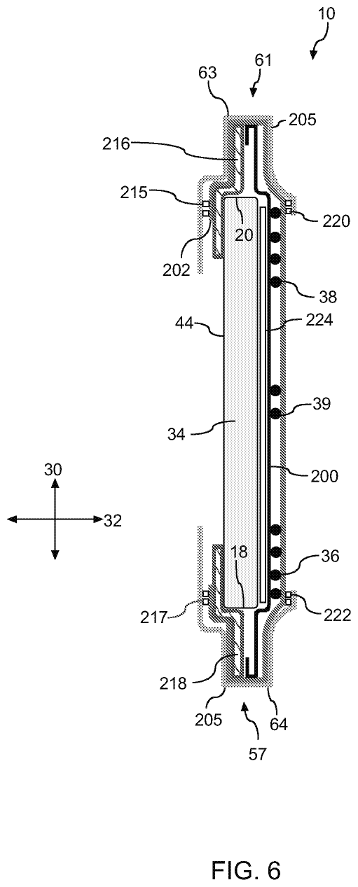

FIG. 6 depicts a longitudinal cross section of an example absorbent garment 10 according to various embodiments of the present disclosure. As shown, the garment 10 includes an absorbent assembly 34 having a skin-facing liner 44 thereon. For example, the absorbent assembly 34 can include a skin-facing liner 44 on the bodyside of the absorbent assembly 34 and be bonded to an inner layer 200 on the opposite side of the absorbent assembly 34. While, one side of the inner layer 200 is bonded to the absorbent assembly 34, the other and opposite side of the inner layer 200 is coupled to one or more elastic areas 36 and 38 disposed between the inner layer 200 and the outer layer 202. To form the back panel 61, a portion of the outer layer 202 is folded over forming a back fold line 63. The folded over portion of the back panel 61 forms the back waist edge of the garment 10. The folded over portion of the back panel 61 is bonded to the skin-facing liner 44 at an attachment region 216. Attachment region 216 is configured such that both a portion of the outer layer 202 and the softened material layer 205 are bonded to the skin-facing liner 44. To form the front panel 57, a portion of the outer layer 202 is folded over forming a front fold line 64. The folded over portion of the front panel 57 forms the front waist edge of the garment 10. The folded over portion of the front panel 57 is bonded to the skin-facing liner 44 at an attachment region 218. Attachment region 218 is configured such that both a portion of the outer layer 202 and the softened material layer 205 are bonded to the skin-facing liner 44.

A softened material layer 205 is disposed on the outer layer 202 such that a portion of the softened material layer 205 is folded over the back fold line 63 and the front fold line 64. For the back panel 61, a portion of the softened material layer 205 can be bonded to the outer layer 202 at an attachment region 220 disposed on the outward facing surface, folded over the back fold line 63, and bonded to the outer layer 202 an attachment region 215 that generally is on the bodyside of the garment 10. Similarly, for the front panel 57, a portion of the softened material layer 205 can be bonded to the outer layer 202 at an attachment region 222 disposed on the outward facing surface of the garment, folded over the front fold line 64, and bonded to the outer layer 202 at an attachment region 217 that is generally on the bodyside of the garment 10. In certain embodiments, the softened material layer 205 remains unbonded from the outer layer 202 except with respect to attachment region 222 and 217. In certain embodiments, as shown in FIG. 6, attachment regions 215 and 217 bond only a portion of the softened material layer to the outer layer 202.

The folded over portion of the back panel 61 can include a softened material layer 205 having an end along the bodyside of the garment that is disposed closer to the central lateral axis (L) as compared to the end of the outer material layer 202 along the bodyside of the garment. Similarly, the folded over portion of the front panel 57 can include a softened material layer 205 having an end along the body side of the garment that is disposed closer to the central lateral axis (L) as compared to the end of the outer material layer 202 along the body side of the garment.

According to some embodiments where an end of the softened material layer 205 along the bodyside of the garment is disposed closer to the central lateral axis (L) as compared to the end of the outer material layer 202 (in either the front and/or back panel 57, 61), portions of the adhesive 218 may be uncovered by the outer material layer 202 and exposed to the softened material layer 205. In such embodiments, the adhesive 218 may bond the softened material layer 205 to the skin-facing liner 44 at a point closer to the central lateral axis L than the end(s) of the outer material layer 202. Attachment regions 215 and 217 may be eliminated in such instances, leaving the softened material layer 205 bonded to the outer material layer 202 at attachment regions 220, 222 and bonded to the skin-facing liner 44 by adhesive 218 and unbonded to the outer material layer 202 between such attachment points.

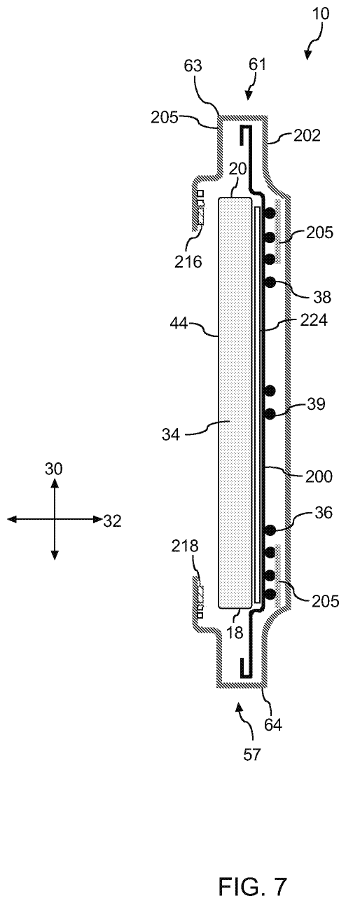

FIG. 7 depicts a longitudinal cross section of an example absorbent garment 10 according to various embodiments of the present disclosure. As shown, the garment 10 includes an absorbent assembly 34 having a skin-facing liner 44 thereon. For example, the absorbent assembly 34 can include a skin-facing liner 44 on the bodyside of the absorbent assembly 34 and be bonded to an inner layer 200 on the opposite side of the absorbent assembly 34. While, one side of the inner layer 200 is bonded to the absorbent assembly 34, the other and opposite side of the inner layer 200 is coupled to one or more elastic areas 36 and 38 disposed between the inner layer 200 and the outer layer 202. To form the back panel 61, a portion of the outer layer 202 is folded over forming a back fold line 63. The folded over portion of the back panel 61 forms the back waist edge of the garment 10. The folded over portion of the back panel 61 is bonded to the skin-facing liner 44 at an attachment region 216. A softened material layer 205 is disposed between the elastic area 38 and the outer layer 202. To form the front panel 57, a portion of the outer layer 202 is folded over forming a front fold line 64. The folded over portion of the front panel 57 forms the front waist edge of the garment 10. The folded over portion of the front panel 57 is bonded to the skin-facing liner 44 at an attachment region 218. A softened material layer 205 is disposed between the elastic area 36 and the outer layer 202.

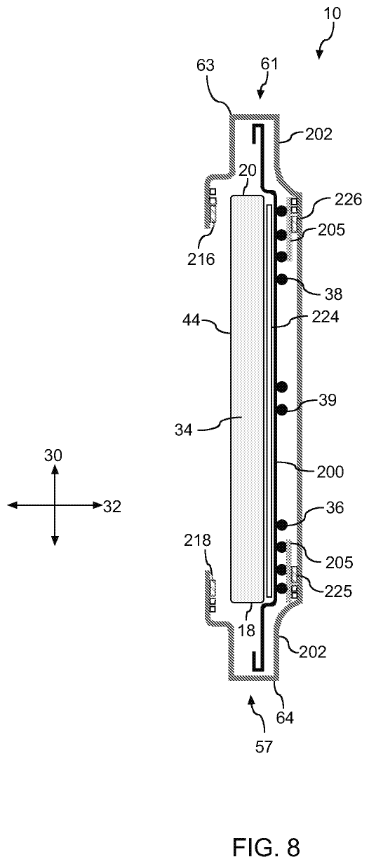

FIG. 8 illustrates the same garment 10 as FIG. 7, except that the garment 10 of FIG. 8 includes one or more attachment regions 225 and 226 for bonding the softened material layer 205 to the outer layer 202.

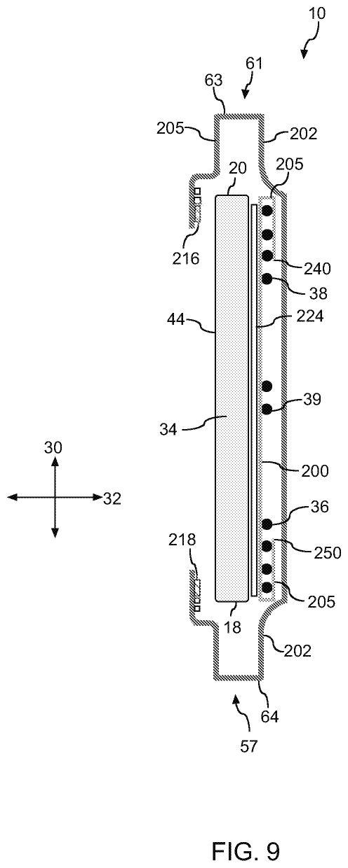

FIG. 9 depicts a longitudinal cross section of an example absorbent garment 10 according to various embodiments of the present disclosure. As shown, the garment 10 includes an absorbent assembly 34 having a skin-facing liner 44 thereon. For example, the absorbent assembly 34 can include a skin-facing liner 44 on the bodyside of the absorbent assembly 34 and be bonded to an inner layer 200 on the opposite side of the absorbent assembly 34. While, one side of the inner layer 200 is bonded to the absorbent assembly 34, the other and opposite side of the inner layer 200 is coupled to one or more elastic areas 36 and 38 disposed between the inner layer 200 and the outer layer 202.

To form the back panel 61, a portion of the outer layer 202 is folded over forming a back fold line 63. The folded over portion of the back panel 61 forms the back waist edge of the garment 10. The folded over portion of the back panel 61 is bonded to the skin-facing liner 44 at an attachment region 216. As shown, the attachment region 216 is configured such only the outer layer 202 is bonded to the skin-facing liner 44 at attachment region 216. The inner layer 200 includes a terminal end 240 comprising the softened material layer 205. The terminal end 240 can be folded over an end of the elastic area 38 to form a folded over portion of the inner layer 200. The folded over portion of the inner layer 200 disposes the terminal end 240 having the softened material layer 205 thereon between the outer layer 202 and the elastic area 38.

To form the front panel 57, a portion of the outer layer 202 is folded over forming the front fold line 64. The folded over portion of the front panel 57 forms the front waist edge of the garment 10. The folded over portion of the front panel 57 is bonded to the skin-facing liner 44 at an attachment region 218. As shown, the attachment region 218 is configured such that only the outer layer 202 is bonded to the skin-facing liner 44 at attachment region 218. The inner layer 200 includes a terminal end 250 comprising the softened material layer 205. The terminal end 250 can be folded over the end of the elastic area 36 to form a folded over portion of the inner layer 200. The folded over portion of the inner layer 200 disposes the terminal end 250 having the softened material layer 205 thereon between the outer layer 202 and the elastic area 36.

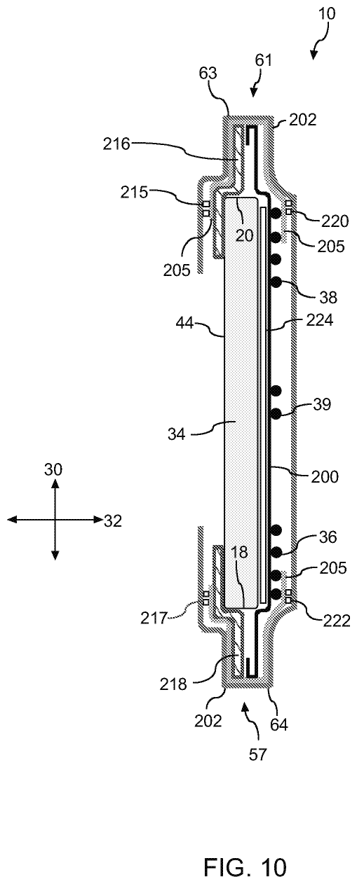

FIG. 10 depicts a longitudinal cross section of an example absorbent garment 10 according to various embodiments of the present disclosure. As shown, the garment 10 includes an absorbent assembly 34 having a skin-facing liner 44 thereon. For example, absorbent assembly 34 can include a skin-facing liner 44 on one side of the absorbent assembly 34 and be bonded to an inner layer 200 on the opposite side of the absorbent assembly 34. While one side of the inner layer 200 is bonded to the absorbent assembly 34, the other and opposite side of the inner layer 200 is coupled to an elastic area 36 and 38 disposed between the inner layer 200 and the outer layer 202. To form the back panel 61, a portion of the outer layer 202 is folded over forming a back fold line 63. The folded over portion of the back panel 61 forms the back waist edge of the garment 10. The folded over portion of the back panel 61 is bonded to the skin-facing liner 44 at an attachment region 216. As shown, attachment region 216 is configured such that the outer layer 202 is bonded to the skin-facing liner 44 and the softened material layer 205 is bonded to the inner layer 200. Similarly, to form the front panel 57, a portion of the outer layer 202 is folded over forming a front fold line 64. The folded over portion of the front panel 56 forms the front waist edge of the garment 10. The folded over portion of the front panel is bonded to the skin-facing liner 44 at an attachment region 218. As shown, attachment region 218 is configured such that outer layer 202 is bonded to the skin-facing liner 44 and the softened material layer 205 is bonded to the inner layer 200.

A softened material layer 205 is disposed on the outer layer 200, such that when the outer layer is folded over to form the back fold line 63, the outer layer fully covers the softened material layer 205 when viewing the body facing side of the garment or the outward facing side of the garment. In such an embodiment, the softened material layer 205 forms one of the internal layers of the back panel 62.

For the back panel 61, the softened material layer 205 is disposed between elastic area 38 and the outer layer 202. As shown, the softened material layer 205 is bonded to the outer layer 202 at attachment region 220. The softened material layer 205 is then folded over and adhered to the outer layer 202 at attachment region 215 along the body facing side of the garment. Similarly, for the front panel 57, the softened material layer 205 is disposed between elastic area 36 and the outer layer 202. The softened material layer 205 is bonded to the outer layer at attachment region 222. The softened material layer 205 is then folded over and adhered to the outer layer at attachment region 217 along the body facing side of the garment. In certain embodiments, the softened material layer 205 remains unbonded from the outer layer 202 except with respect to attachment regions 215, 217, 220, and 222. As shown, in embodiments, attachment regions 215, 217, 220, and 222 only bond a portion of the softened material layer 205 to the outer layer 202.

As shown in FIG. 10, the softened material layer 205 is disposed internally of the outer material layer 202 such that no portion of the softened material layer 205 is in direct contact with the skin of the wearer. The softened material layer 205 is folded in conjunction with the outer layer 202 to provide both the back fold line 63 and the front fold line 64 of the garment. In such an embodiment, the softened material layer 205 provides one or more of the internal layers for the back panel 61 and the front panel 57.

The folded over portion of the back panel 61 can include an outer layer 202 having an end along the bodyside of the garment that is disposed closer to the central lateral axis (L) as compared to the end of the softened material layer 205 on the bodyside of the garment. Similarly, the folded over portion of the front panel (57) can include an outer layer 202 having an end along the body side of the garment that is disposed closer to the central lateral axis L as compared to the end of the softened material layer 205 on the bodyside of the garment. In other embodiments, it is contemplated that the back panel 61 and front panel 57 can include a softened material layer 205 having an end that is disposed closer to the central lateral axis L as compared to the end of the outer layer 202.

It will be appreciated that the inner layer 200 and the outer layer 202 can also form the outer cover 42 for the absorbent assembly 34. For example, as shown in FIGS. 5-9, the absorbent assembly 34 has a skin-facing liner 44 on one side and is bonded to the inner layer 200 on the side opposite the skin-facing liner 44. Thus, both the inner layer 200 and the outer layer 202 span the longitudinal direction of the garment 10 providing material for the outer cover of the absorbent assembly 34 and material for the front waist panel 57 and back waist panel 61. The absorbent assembly 34 is adhered to the inner layer 200 via adhesive region 224. One or more elastic areas, such as elastic areas 36 and 38 can be sandwiched between the inner layer 200 and the outer layer 202. Additionally, other elastic areas, such as a middle elastic area 39 can be placed between the inner layer 200 and the outer layer 202 as shown. Additional elastic areas can be incorporated between the inner layer 200 and the outer layer 202 as needed.

The garments provided herein can be manufactured according to any suitable process. It should be appreciated that embodiments of the present invention are suitable for use with both “machine-direction” and “cross-machine direction” processes, and include those processes for making such garments as disposable diapers, disposable swim pants, disposable training pants, disposable enuresis garments, feminine care products, surgical garments, and the like. Examples of suitable processes are described by U.S. Pat. Nos. 8,491,741 and 8,753,466, both of which are assigned to Kimberly-Clark Worldwide, Inc. and are incorporated by reference herein to the extent not consistent herewith. For example, during manufacturing of the garments disclosed herein, the outer layer 202 and inner layer 200 can both be laid out in a machine direction defining a cross direction width, having a first cross-direction end and a second cross direction end each defining the front and back regions of the garment. An elastomeric material containing the one or more elastic areas 36, 38 can be sandwiched between the inner layer 200 and the outer layer 202. The softened material layer 205 can be disposed on the outer layer 202 or inner layer 200 in desired locations. Individual absorbent assemblies 34 can then be disposed on and bonded to the inner layer 200. The garments 10 can then be cut to form the leg openings 50 and folded to form the front and back waist panels 57 and 61, respectively.

Referring specifically to FIGS. 5, 6, and 10, each of the attachment regions 220 and 215 may be disposed a distance in the longitudinal direction 30 from the top waist edge of the article 10. In some embodiments, the attachment region 220 may be disposed between about 10 mm and about 40 mm from the top waist edge. For example, the back fold line 63 may form the top waist edge along the back of the garment 10. Accordingly, attachment region 220 may be disposed between about 10 mm and about 40 mm from the back fold line 63 defining the top waist edge of the garment. In other embodiments, the attachment region 220 may be disposed between about 10 mm and about 30 mm, or between about 10 mm and about 25 mm, or between about 10 mm and about 20 mm from the top waist edge. Likewise, the attachment region 215 may be disposed between about 10 mm and about 40 mm from the top waist edge, or between about 10 mm and about 30 mm, or between about 10 mm and about 25 mm, or between about 10 mm and about 20 mm from the top waist edge in other embodiments. In further embodiments, however, the attachment regions 220 and 215 may each be disposed a distance in the longitudinal direction 30 from the top waist edge of the article 10 of between about 50 mm and about 150 mm, or between about 50 mm and about 100 mm. Theses embodiments may be more suitable for adult care garments.

In any of these embodiments, a length of softened material layer 205 extends between attachment region 215 and attachment region 220. Indeed, the length of the softened material layer 205 between these attachment regions may be folded over thus forming a portion of the folded over portion of the back panel. Despite being folded, it remains that there is a length of softened material layer 205 that extends between attachment region 215 and attachment region 220. The length of the softened material layer 205 extending between attachment region 215 and attachment region 220, as measured along the softened material layer, may be between about 20 mm and about 80 mm, or between about 20 mm and about 60 mm, or between about 20 mm and about 50 mm, or between about 20 mm and about 40 mm. In still further embodiments, the length of softened material layer 205 that extends between attachment region 215 and attachment region 220 may be between about 100 mm and about 300 mm, or between about 100 mm and about 200 mm. According to some aspects, the softened material layer 205 may be unbonded to the outer layer 202 between the attachment regions 215 and 220. In this manner, the softened material layer 205 may maintain a soft feel and blousy look, expressing a feeling and perception of comfort to both a wearer and one viewing the garment 10.

Each of the attachment regions 222 and 217 may be disposed a distance in the longitudinal direction 30 from the top waist edge of the article 10. In some embodiments, the attachment region 222 may be disposed between about 10 mm and about 40 mm from the top waist edge. For example, the front fold line 64 may form the top waist edge along the front of the garment 10. Accordingly, attachment region 222 may be disposed between about 10 mm and about 40 mm from the front fold line 64 defining the top waist edge of the garment. In other embodiments, the attachment region 222 may be disposed between about 10 mm and about 30 mm, or between about 10 mm and about 25 mm, or between about 10 mm and about 20 mm from the top waist edge. Likewise, the attachment region 217 may be disposed between about 10 mm and about 40 mm from the top waist edge, or between about 10 mm and about 30 mm, or between about 10 mm and about 25 mm, or between about 10 mm and about 20 mm from the top waist edge in other embodiments. In further embodiments, however, the attachment regions 222 and 217 may each be disposed a distance in the longitudinal direction 30 from the top waist edge of the article 10 of between about 50 mm and about 150 mm, or between about 50 mm and about 100 mm. Theses embodiments may be more suitable for adult care garments.

In any of these embodiments, a length of softened material layer 205 extends between attachment region 217 and attachment region 222. Indeed, the length of the softened material layer 205 between these attachment regions may be folded over thus forming a portion of the folded over portion of the front panel. Despite being folded, it remains that there is a length of softened material layer 205 that extends between attachment region 217 and attachment region 222. The length of the softened material layer 205 extending between attachment region 217 and attachment region 222, as measured along the softened material layer, may be between about 20 mm and about 80 mm, or between about 20 mm and about 60 mm, or between about 20 mm and about 50 mm, or between about 20 mm and about 40 mm. In still further embodiments, the length of softened material layer 205 that extends between attachment region 217 and attachment region 222 may be between about 100 mm and about 300 mm, or between about 100 mm and about 200 mm. According to some aspects, the softened material layer 205 may be unbonded to the outer layer 202 between the attachment regions 217 and 222. In this manner, the softened material layer 205 may maintain a soft feel and blousy look, expressing a feeling and perception of comfort to both a wearer and one viewing the garment 10.

In at least some embodiments, it may be the case that a length of the softened material layer 205 extending between the attachment regions 215 and 220 (or between regions 217 and 222), as measured along the softened material layer 205 (termed herein a “softened material length”), may be longer than a distance between the attachment regions 215 and 220 (or between regions 217 and 222) (termed herein a “panel waist region length”), as measured along the front or back panels 57, 61. Measuring along the front and back panels 57, 61 may comprise measuring along the outer layer 202 where the outer layer is folded over forming the front and back fold lines 64, 63 or may comprise measuring along the outer layer 202 from the attachment region 220 (or region 222) to the waist edge and then along the inner layer 200 from the waist edge down to the attachment region 215 (or 217).

According to some aspects of the present disclosure, the softened material length may be at least about 6 mm longer than the panel waist region length. In further embodiments, the softened material length may be at least about 8 mm, or at least about 10 mm, or at least about 12 mm, or at least about 14 mm, or at least about 16 mm greater than the panel waist region length. In still further embodiments, the softened material length may be between about 6 mm and about 20 mm greater than the panel waist region length, or between about 6 mm and about 16 mm, or between about 6 mm and about 14 mm, or between about 6 mm and about 12 mm, or between about 8 mm and about 12 mm greater than the panel waist region length in other embodiments. These differences in lengths may be achieved by varying the tension of the outer and/or inner layers 200, 202 and the softened material layer 205 at the time the softened material layer 205 material is applied to the outer and/or inner layers 200, 202 such that when the tension is released, the outer and/or inner layers 200, 202 retract back to a greater extent than the softened material layer 205, thus realizing a difference in lengths between the attachment regions 215 and 220 (or 217 and 222). Alternatively, the softened material layer 205 may be folded at the time the softened material layer 205 is attached to the outer and/or inner layers 200, 202, or may alternatively form a convex shape at the time of the attachment to the outer and/or inner layers 200, 202.

The folded over portion of both the front panel 57 and the back panel 61 include a softened material layer 205 having an end along the bodyside of the garment 10 that is disposed towards the central lateral axis (L) of the garment 10. In certain embodiments, the softened material layer 205 can include a length of material extending from attachment regions 215, 217 to each respective bodyside end. The length of softened material layer 205 extending from attachment regions 215, 217 to each respective end can be from about 2 mm to about 5 mm, such as about 3 mm, such as about 4 mm. Advantageously, having such a length of softened material layer 205 extending from attachment regions 215, 217 in such a manner avoids adhesive exposure during manufacturing and prevents ruffling of the end of the softened material layer 205 disposed on the body facing side of the garment.

As shown with respect to FIGS. 3-10, the garment 10 may include front and back fold lines 64, 63 whereby a portion of the front and back panels 57, 61 are folded over onto the body-facing side of the garment 10. In further embodiments, however, it is contemplated that the garment 10 may not portions of the front and back panels 57, 61 which fold over onto the body-facing side of the garment 10. In such embodiments, ends of the front and back panels 57, 61 may terminate proximate the front and rear waist edges of the garment 10. In such embodiments, it may only be the softened material layer 205 which is folded over ends of the front and back panels 57, 61 forms the front and back fold lines 64, 63. In these embodiments, the attachment region 215 may bond the softened material layer 205 to the inner layer 200 while the attachment region 220 may bond the softened material layer 205 to the outer layer 202, both regions 215, 220 being disposed on the front panel. Likewise, the attachment region 217 may bond the softened material layer 205 to the inner layer 200 while the attachment region 222 may bond the softened material layer 205 to the outer layer 202, both regions 217, 222 being disposed on the back panel 61.

Each of the attachment regions 215, 216, 217, 218, 220, 222 can include any suitable adhesive. For example, the attachment regions disclosed herein can include an adhesive strip generally having a longitudinal length and a transverse length. The longitudinal length being the length of the strip parallel to the longitudinal direction 30 and the transverse length being the length of the strip parallel to the transverse direction 32. In embodiments, the strip will have a longitudinal length that is less than the transverse length. In certain embodiments the longitudinal length of the adhesive strip may be from about 1 mm to about 40 mm, such as from about 2 mm to about 30 mm, such as from about 3 mm to about 20 mm. In certain embodiments, the attachment regions can include one or more strips of adhesive. In such embodiments where multiple adhesive strips are desirable, the adhesive strips my have the same or different longitudinal lengths.

Application of adhesives to absorbent articles, such as diapers, can affect the elasticity and the softness of the underlying layers of material. Furthermore, application of adhesive strips under body facing material layers have the potential to cause additional irritation to the skin of the wearer or can reduce the softness of the material layers. Additionally, application of adhesives under certain softened materials can bleed through the material layer, which is undesirable. Accordingly, attachment regions 215 and 217 can include an adhesive strip having a longitudinal length of from about 0.5 mm to about 5 mm, such as from about 1 mm to about 4 mm, such as from about 2 mm to about 3 mm. Such an adhesive strip having a reduced longitudinal length can prevent irritation and bleed through from the adhesive. Additionally, having such a narrow strip of adhesive as described for attachment regions 215 and 217, may allow for higher attachment strength along attachment regions 215,217 as compared to other attachment regions having a larger surface area in the longitudinal direction. Additionally, portions of the softened material layer 205 may remain unbonded to except with respect to the attachment regions described herein. Accordingly, attachment regions 215 and 217 having the described longitudinal lengths allow for the softened material layer to remain untethered to portions of the front and back panels 57,61 such that softness provided by the softened material layer 205 is not disrupted with the use of additional adhesives.

In certain embodiments, attachment regions 216 and 218 can include an adhesive strip having a longer longitudinal length as compared to attachment regions 215 and 217. For example, attachment regions 216 and 218 can include one or more adhesive strips having a longitudinal length of from about 15 mm to about 45 mm, such as from about 20 mm to about 30 mm. Providing an adhesive strip having the longitudinal lengths disclosed herein at attachment regions 216 and 218 ensures that the absorbent assembly 34 is properly bonded to the outer layer 202 such that the folded over portion of the back panel 61 or the front panel 57 does not come undone from the absorbent assembly 34.

In addition to the description of different aspects of the present invention already provided, further description of the individual components of the absorbent garment 10 is helpful. For example, the softened material layer 205 can be a single-layer or multi-layered material and can be selected from nonwoven materials, laminates, spunbond fabrics, spun-melt fabrics (including spunbond-meltblown-spunbond (SMS) fabrics and other similar laminate fabrics), films, meltblown fabrics, elastic netting, microporous web, bonded carded webs (included through air bonded carded webs, abbreviated TABCW herein), surge materials, or foams comprised of elastomeric or polymeric materials or cellulosic materials such as tissue. In embodiments, the softened material layer includes a nonwoven fabric or web. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns, although denier is sometimes used to refer to fiber thickness. For example, in certain embodiments, the softened material layer may have a basis weight of between about 7 gsm and about 45 gsm, or between about 7 gsm and about 35 gsm, or between about 7 gsm and about 25 gsm, or between about 10 gsm and about 20 gsm In such embodiments, a preferred type of material for the softened material layer 205 may be a spunbond, spunmelt, or TABCW material.

In particular embodiments where the softened material layer includes a spunbond fabric having one or more spunbonded fibers therein, the spunbond fibers may be formed from any suitable polymer material. The softened material layer can be formed from any suitable thermoplastic polymer. For example, thermoplastic polymers which may be used may be any known to those skilled in the art to be commonly used in meltblowing and spunbonding. Such polymers include polyolefins, polyesters, polyetherester, polyurethanes and polyamides, and mixtures thereof, more particularly polyolefins such as polyethylene, polypropylene, polybutene, ethylene copolymers, propylene copolymers and butene copolymers.

In certain embodiments the softened material layer can comprise a single layer or a multilayer laminate. Such a multilayer laminate can include embodiments wherein some of the layers are spunbond and some of the layers are meltblown, such as a spunbond/meltblown/spunbond (SMS) laminates. Examples of such SMS laminates are disclosed in U.S. Pat. No. 4,041,203 to Brock et al. and U.S. Pat. No. 5,169,706 to Collier, et al. Such a laminate may be made by sequentially depositing onto a moving forming belt first a spunbond fabric layer, then a meltblown fabric layer and last another spunbond layer and then bonding the laminate according to known methods. Alternatively, the fabric layers may be made individually, collected in rolls, and combined in a separate bonding step. Such fabrics usually have a basis weight of from about 6 to about 400 grams per square meter. The process of this invention may also produce fabric which has been laminated with films, glass fibers, staple fibers, paper, and other web materials.

The softened material layer 205 can be composed substantially of a nonwoven web of material that has been treated with a softener to impart a desired level of soft texture to the material layer. For example, the softened material layer 205 can be treated with one or more softener additives, such as softening chemicals. For example, softening chemicals can be added in various amounts to the nonwoven web in order to provide the softened material layer 205. Softening chemicals may be any of those commonly known to those skilled in the art as being useful for softening textiles. Softeners may be silicone, anionic, nonionic or cationic.

Anionic softeners are generally chemical compounds such as sulfated oils like castor, olive and soybean, sulfated synthetic fatty esters, such as glyceryl trioleate, and sulfated fatty alcohols of high molecular weight.

Nonionic softeners are highly compatible with other finishing agents and are generally compounds such as glycols, glycerin, sorbitol and urea. Compounds of fatty acids like polyglycol esters of high molecular weight saturated fatty acids such as palmitic and stearic acids are other examples.

Cationic softeners are generally long chain amides, imidazolines, and quarternary nitrogen compounds. One suitable cationic softener is a tallow based quarternary ammonium compound sold under the tradename Varisoft®.

In certain embodiments, the nonwoven web of the softened material layer 205 can be subjected to any known mechanical process for softening a nonwoven web. Examples of chemical and mechanical softening processes for nonwoven webs are disclosed in U.S. Pat. No. 5,413,811 to Fitting, et al.

In other embodiments, the softened material layer 205 can be composed substantially of a nonwoven web of material comprising a polymer composition that includes one or more polymers capable of imparting softness to the nonwoven web. For example, the softened material layer 205 can be composed of a polymer composition containing Vistamaxx® performance polymers available ExxonMobil. In embodiments, the polymer composition used to form the nonwoven web of material can include from about 1% w/w to about 7.5% w/w of Vistamaxx® performance polymers such as from about 1% w/w to about 5% w/w, or from about 1% w/w to about 4% w/w, from about 1% w/w to about 3.5% w/w, such as about 3.5% w/w, or from about 1% w/w to about 2.5% w/w. It has been found that going above 3.5%, or 5%, or 7.5% in certain embodiments, while making the softened material layer 205 softer, prevents the softened material layer 205 from bonding with itself or other materials, thus making constructing of article 10 very difficult or impossible. Other performance polymers or polymer blends can also be added to the polymer composition used to form the softened material layer 205.

Alternatively, or additionally, to adding one or more softener additives to the nonwoven material forming the softened material layer 205 to ensure a desired level of softness, it may be desirable to ensure the deniers of the fibers forming the softened material layer 205 are sufficiently small. It has been found that deniers of the fibers forming the softened material layer are preferably less than about 2.0, or more preferably less than about 1.7, or more preferably less than about 1.5, or more preferably less than about 1.3. or more preferably less than about 1.2. Smaller denier fibers generally correlate to a softer feeling material.

Furthermore, the absorbent assembly 34 is configured to contain and/or absorb bodily wastes discharged from the wearer. Accordingly, the outer cover 42 of the garment 10 suitably includes a material which is substantially liquid impermeable, i.e. a film layer. In certain embodiments it should be appreciated that the outer cover 42 of the absorbent assembly can be comprised of a multi-layered material comprising the inner layer 200 and the outer layer 202. For example, the absorbent composite 46 can be bonded to the inner layer 200 and a skin-facing liner 44 can then be applied over the absorbent composite 46. In such embodiments, the inner layer 200 can be a liquid impermeable layer and the outer layer 202 can be a liquid permeable layer. The liquid permeable outer layer 202 and a liquid impermeable inner layer 200 can be joined together by an adhesive, ultrasonic bonding, thermal bonding, pressure bonding, or combinations thereof. Suitable adhesives can be applied continuously or intermittently as beads, a spray, parallel swirls, or the like. The liquid permeable outer layer 202 can be any suitable material, including materials that provide a generally cloth-like texture. The outer layer 202 can also be made of those materials of which the liquid permeable skin-facing liner 44 is made. While it is not a necessity for the outer layer 202 to be liquid permeable, it is suitable that it provides a relatively cloth-like texture to the wearer.

The inner layer 200 can be both liquid and vapor impermeable, or it can be liquid impermeable and vapor permeable. The inner layer 200 can be manufactured from a thin plastic film, although other flexible liquid impermeable materials can also be used. The inner layer 200 can prevent waste material from wetting articles, such as bed sheets and clothing, as well as the wearer and caregiver.

The outer layer 202 can be embossed and/or matte finished to provide a more cloth-like appearance. As earlier mentioned, the liquid impermeable material can permit vapors to escape from the interior of the absorbent garment, while still preventing liquids from passing through the inner layer 200 and the outer layer 202. One suitable “breathable” material is composed of a microporous polymer film or a nonwoven fabric that has been coated or otherwise treated to impart a desired level of liquid impermeability. It is also contemplated that the inner layer 200 and outer layer 202 can be stretchable, and more suitably elastic. In particular, the inner layer 200 and outer layer 202 are suitably stretchable and more suitably elastic in at least the transverse (lateral), or circumferential direction of the absorbent garment 10. In other aspects the inner layer 200 and outer layer 202 can be stretchable, and more suitably elastic, in both the transverse (lateral) 32 and the longitudinal direction 30.

The liquid permeable skin-facing liner 44 overlies the absorbent composite 46, and can, but need not, have the same dimensions as the inner layer 200. In certain embodiments, the skin-facing liner 44 overlies the absorbent composite 46 and is bonded to the inner layer 200 along the transverse ends of the absorbent composite 46. The skin-facing liner 44 can be bonded to the inner layer 200 via any suitable bonding mechanism.

The skin-facing liner 44 is suitably compliant, soft feeling, and non-irritating to the wearer's skin. The skin-facing liner 44 is also sufficiently liquid permeable to permit liquid body exudates to readily penetrate through its thickness to the absorbent composite 46. Further, the skin-facing liner 44 can be less hydrophilic than the absorbent composite 46 to present a relatively dry surface to the wearer and permit liquid to readily penetrate through its thickness. The hydrophilic/hydrophobic properties can be varied across the length, width and/or depth of the skin-facing liner 44 and absorbent composite 46 to achieve the desired wetness sensation or leakage performance.

The skin-facing liner 44 can be manufactured from a wide selection of web materials, such as porous foams, reticulated foams, apertured plastic films, woven and nonwoven webs, or a combination of any such materials. For example, the skin-facing liner 44 can include a meltblown web, a spunbonded web, or a bonded-carded-web composed of natural fibers, synthetic fibers or combinations thereof. The skin-facing liner 44 can be composed of a substantially hydrophobic material, and the hydrophobic material can optionally be treated with a surfactant or otherwise processed to impart a desired level of wettability and hydrophilicity. The surfactant can be applied by any conventional means, such as spraying, printing, brush coating or the like. The surfactant can be applied to the entire skin-facing liner 44 or can be selectively applied to particular sections of the skin facing liner 44, such as the medial section along the longitudinal center line.

The skin-facing liner 44 can also be stretchable, and, more suitably, it can be elastomeric. In particular, the skin-facing liner 44 is suitably stretchable and more suitably elastomeric in at least the lateral or circumferential direction 32 of the absorbent garment 10. In other aspects, the skin-facing liner 44 can be stretchable, and more suitably elastomeric, in both the lateral direction 32 and the longitudinal direction 30.

The absorbent composite 46 is suitably compressible, conformable, non-irritating to a wearer's skin, and capable of absorbing and retaining liquids and certain body wastes. For example, the absorbent composite 46 can include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. The materials can be formed into a web structure by employing various conventional methods and techniques. For example, the absorbent composite 46 can be formed by a dry-forming technique, an air forming technique, a wet-forming technique, a foam-forming technique, or the like, as well as combinations thereof. Furthermore, the absorbent composite 46 can itself encompass multiple layers in a Z-direction (e.g., thickness) of the absorbent composite 46. Such multiple layers can take advantage of differences in absorbent capacity, such as by placing a lower absorbent capacity material layer closer to the skin-facing liner 44 and a higher absorbent capacity material closer to the outer cover 42. Likewise, discrete portions of a single-layered absorbent composite can encompass higher capacity absorbents, and other discrete portions of the composite can encompass lower capacity absorbents.

Superabsorbent material is suitably present in the absorbent composite 46 in an amount of from about 0 to about 100 weight percent based on total weight of the absorbent composite 46. The absorbent composite 46 can suitably have a density within the range of about 0.10 to about 0.60 grams per cubic centimeter. Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and preferably is capable of absorbing more than about 25 times its weight in liquid.

The absorbent composite 46 can alternatively include a coform material. The term “coform material” generally refers to composite materials including a mixture or stabilized matrix of thermoplastic fibers and a second non-thermoplastic material. As an example, coform materials are made by a process in which at least one meltblown die head is arranged near a chute through which other materials are added to the web while it is forming. Such other materials can include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers can be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene.

In one suitable aspect, the absorbent composite 46 is stretchable so as not to inhibit the stretchability of other components to which the absorbent composite 46 can be adhered, such as the outer cover 42 and the skin-facing liner 44. After being formed or cut to a desired shape, the absorbent composite 46 can be wrapped or encompassed by a suitable wrap (not shown) that aids in maintaining the integrity and shape of the absorbent composite 46.

The absorbent assembly 34 can also include a surge management layer (not shown) located adjacent the absorbent composite 46 (e.g., between the absorbent composite 46 and the skin-facing liner 44) to help decelerate and diffuse surges or gushes of liquid that can be rapidly introduced into the absorbent composite 46 of the absorbent garment 10 by the wearer. Desirably, the surge management layer can rapidly accept and temporarily hold the liquid prior to releasing the liquid into the storage or retention portions of the absorbent composite 46. Examples of suitable surge management layers are described in U.S. Pat. No. 5,486,166 issued Jan. 23, 1996 to Bishop et al.; U.S. Pat. No. 5,490,846 issued Feb. 13, 1996 to Ellis et al.; and U.S. Pat. No. 5,820,973 issued Oct. 13, 1998 to Dodge, II et al., the entire disclosures of which are hereby incorporated by reference.

As already described in relation to the various aspects of the present invention, the absorbent assembly 34 can include a pair of containment flaps (not shown) that extend longitudinally along the absorbent assembly 34 and are adapted to provide a barrier to the lateral flow of body exudates as is known in the art. The containment flaps can be connected to the skin-facing liner 44 or other components of the absorbent assembly 34. Suitable configurations of the containment flaps are described, for example, in U.S. Pat. No. 5,599,338 issued Feb. 4, 1997, to K. Enloe, the entirety of which is incorporated herein by reference.

The elastic areas 36,38 are attached to at least one of the inner layer and the outer layer to impart a desired level of elasticity to the front and back panels 57,61. The elastic areas 36,38 may be attached to the inner layer 200 and/or the outer layer 202 by any suitable means including, for example, adhesives. The elastic areas 36,38 may be stretched and then adhered to one or both of the inner layer 200 and the outer layer 202, or adhered to one or both of the inner layer 200 and the outer layer 202 when the layers are in a gathered state to impart a desired level of elasticity to the front panel 57 and back panel 61. In other aspects, the elastic areas 36,38 may be adhered to one or both of the inner layer 200 and the outer layer 202, and then elasticized or shrunk, for example with the application of heat, such that elastic retractive forces are imparted to the front panel 57 and back panel 61.

The elastic areas 36,38 may be formed of a variety of suitable elastic materials, including strands or ribbons of natural rubber, synthetic rubber, or thermoplastic elastomeric polymers. In the aspect shown in FIG. 1, the elastic areas 36,38 includes a plurality of elastomeric strands extending in the lateral direction 32 (FIG. 1) or circumferential direction (FIG. 2) of the absorbent garment 10. In one suitable aspect, the elastomeric strands include dry-spun coalesced multifilament spandex elastomeric strands sold under the trade name LYCRA® and available from Invista of Wichita, Kans., U.S.A. Other suitable materials from which the elastic area 36 may be constructed include vertical filament laminate (VFL) materials, an example of which is described in U.S. Pat. No. 6,916,750 to Thomas et al., which is hereby incorporated by reference.

The inner layer 200 and the outer layer 202 can be joined together in face-to-face relationship by suitable means such as adhesives, ultrasonic bonding, thermal bonding, pressure bonding, or combinations thereof. Suitable adhesives, which can be applied continuously or intermittently as beads, a spray, parallel swirls, or the like, include elastomeric adhesives (i.e. materials capable of at least 75% elongation without rupture), such as aqueous-based styrene butadiene adhesives, neoprene, polyvinyl chloride, vinyl copolymers, polyamides, and ethylene vinyl terpolymers.

In the interests of brevity and conciseness, any ranges of values set forth in this disclosure contemplate all values within the range and are to be construed as support for claims reciting any sub ranges having endpoints that are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value.

For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by references, the meaning or definition assigned to the term in this written document shall govern.

While particular aspects of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed:

1. An absorbent garment comprising:
a longitudinal direction, a lateral direction, and a central lateral axis;
a front region, a back region, a crotch region, the crotch region being disposed between the front region and the back region, the crotch region including an absorbent assembly;
the absorbent assembly including a skin-facing liner and an absorbent composite, the absorbent assembly also includes a front edge, a back edge and a pair of laterally-opposed side edges;
a front panel in the front region, wherein the front panel includes an inner layer, an outer layer, a softened material layer, and an elastic area disposed between the inner layer and the outer layer, a portion of the front panel being folded over forming a front waist edge about a front fold line, wherein a portion of the softened material is folded over the front fold line, wherein the folded over portion of the front panel includes a first attachment region bonding the outer layer to the skin-facing liner and a second attachment region bonding the softened material layer to the outer layer,
a back panel in the back region, wherein the back panel includes an inner layer, an outer layer, a softened material layer, and an elastic area disposed between the inner layer and the outer layer, a portion of the back panel being folded over forming a back waist edge about a back fold line, wherein a portion of the softened material is folded over the back fold line, wherein the folded over portion of the back panel includes a third attachment region bonding the outer layer to the skin-facing liner and a fourth attachment region bonding the softened material layer to the outer layer; and
the front panel and the back panel are bonded to each other to form side seams of the absorbent garment.

2. The garment of claim 1, wherein the portion of the softened material layer in the folded over portion of the front panel has an end that is disposed closer to the central lateral axis as compared to an end of the outer layer in the folded over portion of the front panel, and wherein the portion of softened material layer in the folded over portion of the back panel has an end that is disposed closer to the central lateral axis as compared to an end of the outer layer in the folded over portion of the back panel.

3. The garment of claim 1, wherein the portion of the outer layer in the folded over portion of the front panel has an end that is disposed closer to the central lateral axis as compared to an end of the softened material layer in the folded over portion of the front panel, and wherein the portion of the outer layer in the folded over portion of the back panel has an end that is disposed closer to the central lateral axis as compared to an end of the softened material layer in the folded over portion of the back panel.

4. The garment of claim 1, wherein the second attachment region and fourth attachment region include an adhesive for bonding the softened material layer to the outer layer.

5. The garment of claim 4, wherein the adhesive includes at least one strip of adhesive having a longitudinal length and a transverse length, wherein the longitudinal length is from about 1 mm to about 4 mm.

6. The garment of claim 1, wherein the first attachment region and the third attachment region include an adhesive for bonding the outer layer to the skin-facing liner.

7. The garment of claim 1, wherein the first attachment region and the third attachment region bond a portion of the softened material layer to the skin-facing liner.

8. The garment of claim 6, wherein no portion of the adhesive of the first attachment region or the third attachment region contacts the softened material layer.

9. The garment of claim 1, wherein where in the front panel includes a fifth attachment region bonding the softened material layer to a portion of the front panel opposite the folded over portion and the back panel includes a sixth attachment region bonding the softened material layer to the a portion of the back panel opposite the folded over portion.

10. The garment of claim 1, wherein the second attachment region is located from about 10 mm to about 40 mm from the front waist edge and, wherein the fourth attachment region is located from about 10 mm to about 40 mm from the back waist edge.

11. The garment of claim 9, further comprising a first length of softened material layer extending between the second attachment region and the fifth attachment region and being unbonded between the second attachment region and the fifth attachment region and a second length of softened material layer extending between the fourth attachment region and the sixth attachment region and being unbonded between the fourth attachment region and the sixth attachment region, the first length and the second length each being from about 20 mm to about 80 mm.

12. An absorbent garment comprising:
a longitudinal direction and a lateral direction;
a front region, a back region, a crotch region, the crotch region being disposed between the front region and the back region, the crotch region including an absorbent assembly;
the absorbent assembly including a skin-facing liner and an absorbent composite, the absorbent assembly also includes a front edge, a back edge and a pair of laterally-opposed side edges;
a front panel in the front region, wherein the front panel includes an inner layer, an outer layer, a softened material layer, and an elastic area, wherein the elastic area is disposed between the inner layer and the softened material layer, wherein the softened material layer is disposed between the outer layer and the elastic area, a portion of the front panel being folded over to form a front waist edge, wherein the front panel includes a first attachment region bonding the outer layer to the skin-facing liner and a second attachment region bonding the softened material layer to the outer layer;
a back panel in the back region, wherein the back panel includes an inner layer, an outer layer, a softened material layer, and an elastic area, wherein the elastic area is disposed between the inner layer and the softened material layer, wherein the softened material layer is disposed between the outer layer and the elastic area, a portion of the back panel being folded over to form a back waist edge, wherein the back panel includes a third attachment region bonding the outer layer to the skin-facing liner and a fourth attachment region bonding the softened material layer to the outer layer; and
the front panel and the back panel are bonded to each other to form side seams of the absorbent garment.

13. The garment of claim 12, wherein the second attachment region and fourth attachment region include an adhesive for bonding the softened material layer to the outer layer and wherein the adhesive includes at least one strip of adhesive having a longitudinal length and a transverse length, wherein the longitudinal length is from about 1 mm to about 4 mm.

14. The garment of claim 13, wherein the adhesive includes at least two strips of adhesive having different longitudinal lengths.

15. The garment of claim 13, wherein the folded over portion of the front panel includes the softened material layer and the folded over portion of the back waist panel includes the softened material layer.

16. The garment of claim 15, wherein the portion of the softened material layer in the folded over portion of the front panel has an end that is disposed closer to the central lateral axis as compared to an end of the outer layer in the folded over portion of the front panel, and wherein the portion of softened material layer in the folded over portion of the back panel has an end that is disposed closer to the central lateral axis as compared to an end of the outer layer in the folded over portion of the back panel.

17. The garment of claim 15, wherein the portion of the softened material layer in the folded over portion of the front panel has an end that is disposed further away from the central lateral axis as compared to an end of the outer layer in the folded over portion of the front panel, and wherein the portion of softened material layer in the folded over portion of the back panel has an end that is disposed further away from the central lateral axis as compared to an end of the outer layer in the folded over portion of the back panel.

18. The garment of claim 17, wherein the first attachment region and the third attachment region include an adhesive for bonding a portion of the softened material layer to the skin-facing liner and a portion of the outer layer to the skin-facing liner.

19. The garment of claim 12, wherein the softened material layer comprises a nonwoven material treated with a softener additive.

20. An absorbent garment comprising:
a longitudinal direction and a lateral direction;
a front region, a back region, a crotch region, the crotch region being disposed between the front region and the back region, the crotch region including an absorbent assembly;
the absorbent assembly including a skin-facing liner and an absorbent composite, the absorbent assembly also includes a front edge, a back edge and a pair of laterally-opposed side edges;
a front panel in the front region, wherein the front panel includes an inner layer, an outer layer, and an elastic area disposed between the inner layer and the outer layer, wherein the inner layer includes a first end comprising a softened material layer, wherein the first end including the softened material layer is folded over the elastic area such that the softened material layer forms a folded over portion disposed between the elastic area and the outer layer, a portion of the front panel being folded over to form a front waist edge, wherein the front panel includes a first attachment region bonding the outer layer to the skin-facing liner;
a back panel in the back region, wherein the back panel includes an inner layer, an outer layer, and an elastic area disposed between the inner layer and the outer layer, wherein the inner layer includes a second end comprising a softened material layer, wherein the second end including the softened material layer is folded over the elastic area such that the softened material layer forms a folded over portion disposed between the elastic area and the outer layer, a portion of the back panel being folded over to form a back waist edge, wherein the back panel includes a second attachment region bonding the outer layer to the skin-facing liner; and
the front panel and the back panel are bonded to each other to form side seams of the absorbent garment.

21. The garment of claim 9, wherein a length of the softened material layer extending between the second attachment region and the fifth attachment region, as measured along the softened material layer, is longer than a distance between the second attachment region and the fifth attachment region, as measured along the front panel.

22. The garment of claim 21, wherein the length of the softened material layer extending between the second attachment region and the fifth attachment region, as measured along the softened material layer, is longer than a distance between the second attachment region and the fifth attachment region, as measured along the front panel by at least 6 mm.

23. The garment of claim 12, wherein the front panel includes a fifth attachment region bonding the softened material layer to a portion of the front panel opposite the folded over portion, and wherein a length of the softened material layer extending between the second attachment region and the fifth attachment region, as measured along the softened material layer, is longer than a distance between the second attachment region and the fifth attachment region, as measured along the front panel.

24. The garment of claim 23, wherein the length of the softened material layer extending between the second attachment region and the fifth attachment region, as measured along the softened material layer, is longer than a distance between the second attachment region and the fifth attachment region, as measured along the front panel by at least 6 mm.
